(19) United States
(12) Patent Application Publication
(10) Pub. No.: US 2005/0074407 A1
(43) Pub. Date: Apr. 7, 2005

(54) PVP and PVA as in vivo biocompatible acoustic coupling medium

(75) Inventor: Larry L. Smith, Lummi Island, WA (US)

Correspondence Address: Robert L. McDowell, 1170 Jackson Heights Drive, Webster, NY, 14580, US

(73) Assignee: SONOTECH, INC.

(21) Appl. No.: 10/951,220

(22) Filed: Sep. 27, 2004

Related U.S. Application Data
(60) Provisional application No. 60/507,840, filed on Oct. 1, 2003.

(51) Int. Cl. A61K 49/00; A61K 9/14

(52) U.S. Cl. 424/9.5; 424/484

(57) ABSTRACT

An vivo biocompatible and bio-excretable lubricant and ultrasound coupling fluid or gel comprising polyvinylpyrrolidone (PVP) and/or polyvinyl alcohol (PVA). The inventive couplant fluid or gel comprises polyvinylpyrrolidone and/or polyvinyl alcohol solutions in water to which humectants such as alkylene glycols and/or polyalkylene glycols are added to achieve desired tactile and drying characteristics. Additionally, such fluids and gels may be prepared by addition of organic and inorganic cross-linkers.

This application claims the benefit of U.S. Provisional Patent Application No. 60/507,840 filed Oct. 1, 2003.

### FIELD OF THE INVENTION

The present invention is directed toward the medical use of polyvinylpyrrolidone (PVP) and/or polyvinyl alcohol (PVA) as an in vivo biocompatible acoustic coupling gel and instrument lubricant for use in ultrasound imaging, doppler based flow measurement, and High Intensity Focused Ultrasound (HI FU) therapy when performed inside the body, such as during surgery and with invasive procedures.

### BACKGROUND OF THE INVENTION

Medical applications of ultrasound generally utilize electromagnetic wave frequencies which typically range between 0.5 and 20 MHz for imaging, High Intensity Focused Ultrasound (HIFU) therapy and flow measurements. Ultrasound energy at these frequencies is poorly transmitted by air, which therefore, requires a coupling or conduction medium that possesses acoustic properties similar to tissue and organs. Such media can consist of fluids, gels and certain solid materials and films, to transfer the acoustic energy between the body and the electronics of the diagnostic instrument. This media is commonly referred to as an ultrasound couplant, ultrasound gel, ultrasound transmission media or acoustic transmission media. Many fluids and water-based gels have been used as ultrasound couplants over the years. Early use of mineral oil was replaced by gels of water and acrylic based polymers such as CARBOPOL®, (a registered trademark of BF Goodrich Specialty Chemicals), typical of those such as described in U.S. Pat. No. 4,002,221 to Buchalter, and also gels made from acrylic polymers and attached as coupling members to transducers, such as are described in U.S. Pat. No. 4,459,854 to Richardson et al. as a method for improvement of perivascular blood flow measurement.

The materials and methods described above are known to be utilized when transferring and coupling ultrasound energy between the active face of an ultrasound transducer or suitable acoustic standoff or delay line and the human or animal body. However, such ultrasound coupling fluids and gels, when used in surgical, and ultrasound guided needle puncture procedures, have fundamental disadvantages that place the patient at risk. Some of these disadvantages are described below:
1. Oils or thickened water-based gels typically used in medical ultrasound are similarly described as in previously discussed U.S. Pat. No. 4,002,221, and are comprised of compounds such as acrylic polymers, carboxy alkyl cellulose, hydroxyethylcellulose, carboxy polymethylene, polyalkylene glycol humectants, organic acids, alkali metal salts, parabens and other germicidal and fungicidal agents, and surfactants that are unsuitable for use in applications where they may be carried into the body tissue or fluids.
2. The above-mentioned couplants are also commercially available in sterilized form, thus implying and encouraging their inappropriate use in vivo where their chemical constituents are known to either be harmful to the human body or have not been evaluated for their in vivo use.
3. Currently available ultrasound couplants supplied in sterile form contain acrylic polymers such as CARBOPOL as a common and primary ingredient. CARBOPOL, for example, has not been tested for in vivo biocompatibility. Some currently available sterile couplants also contain cellulose ethers to increase salt stability. According to E. Doecker in “Water Swollen Cellulose Derivatives in Pharmacy” from Hydrogels in Medicine and Pharmacy: Vol. 2-Polymers, edited by Peppas N. A., CRC Press Inc., Boca Raton, Fla., 1987, pg. 124, “In common use, such celluloses are used orally and externally; however, parenteral administration of cellulose is not recommended since derivatives are not easily metabolized”. Since various chemicals of these formulations are not in vivo biocompatible, they can remain in the body as substances that can cause inflammation, disruption in flow of lymph, irritation, anaphylactic shock and other immune system reactions. This concern becomes apparent during ultrasound guided needle biopsy or aspiration, or when ultrasound transducers are used inside the body, for imaging during surgery, in contact with organs, tissue and blood.
4. Of additional concern are the unknown chemical constituents formed during sterilization processing. Methods of couplant sterilization include steam autoclave, E-beam, broad spectrum light and gamma radiation protocols. Couplant products that incorporate CARBOPOL in the formulation can break down due to heat during the autoclave cycle. When exposed to ionizing radiation, such as in the case of gamma, E-beam, and high intensity light sterilization, free radicals can be formed which initiate chain scission and cross linking of the polymer, as evidenced by presence of bubbles and changes in color, viscosity and mechanical properties of the polymer products.
5. It is important to note that sterility of a substance does not guarantee that it is biocompatible, or of greater importance, in vivo biocompatible. When a substance is sterile, it does not contain live microorganisms; however, such sterile materials may not be in vivo-biocompatible should they contain compounds that are incompatible with tissue or body fluids. For example, natural and synthetic materials that are recognized by the FDA as GRAS (Generally Regarded As Safe) may not be in vivo biocompatible. An in vivo biocompatible substance is both sterile, containing no living micro-organisms, and contains no chemicals or substances that are toxic or cause inflammation or immune system reactions, such as from pyrogens, within the living human body. A substance such as the device of this invention is in vivo biocompatible as an ultrasound couplant in contact with human tissue and body fluids.

U.S. Pat. No. 6,302,848 to Larson, et al. describes an ultrasound coupling gel that is in vivo biocompatible and degradable in vivo, consisting of water, propylene glycol and polyethylene oxide of various molecular weights. However, Larson speaks neither to the use of PVP or PVA gels, in cross-linked forms, nor to formulations which contain PVP or PVA gelled with plasticizers or to the application of such polymer formulations as in vivo biocompatible ultrasound couplants that can be eliminated from the body through natural pathways and processes.

U.S. Pat. No. 5,575,291 to Hayakawa describes a production technique to form gel that involves repeated freeze thaw cycles of PVA solutions to create a solid ultrasound coupler and standoff. The method involves injection of a 3 to 6% aqueous solution PVA, having a degree of saponification of not less than 98%, into a mold and subjected to one or more freeze-thaw cycles to form a solid. The device of Hayakawa is a solid and requires attachment of the coupling member to an ultrasound probe for use.

The formulations of the device of the present invention provide ultrasound couplants that have superior rheology and tactile characteristics, are easily applied and removed from patients and instrumentation, yet impart required ultrasound transmission characteristics.

It is an object of the present invention to provide ultrasound couplants and device lubricants for use in all medical ultrasound applications where such formulations may contact body tissue, fluids and organs and when used as a lubricant to facilitate the passage of imaging devices into body cavities.

It is a further object of the present invention to provide gels and fluids that are in vivo biocompatible, and suitable for use in medical diagnostic and therapeutic ultrasound procedures that are invasive to the body of a human during surgery, guided biopsy, within body cavities and ophthalmic imaging.

### SUMMARY OF THE INVENTION

The present invention is directed to an vivo biocompatible and bio-excretable lubricant and ultrasound coupling fluid or gel comprising polyvinylpyrrolidone (PVP) and/or polyvinyl alcohol (PVA). The inventive couplant fluid or gel comprises polyvinylpyrrolidone and/or polyvinyl alcohol solutions in water to which humectants such as alkylene glycols and/or polyalkylene glycols are added to achieve desired tactile and drying characteristics. Additionally, such fluids and gels may be prepared by addition of organic and inorganic cross-linkers.

### DETAILED DESCRIPTION OF THE PREFERRED EMBODIMENTS

The present invention is directed toward the medical use of acoustic coupling fluids and gels used in vivo ultrasound imaging, doppler based flow measurement and in ultrasound guided transcutaneous biopsy and in High Intensity Focused Ultrasound (HIFU) therapy.

The present invention is a medical device lubricant and ultrasound coupling media in gel or liquid form, comprised of polyvinylpyrrolidone (PVP) and/or polyvinyl alcohol (PVA), preferably polyvinylpyrrilidone, blended with water, humectants and plasticizers such as alkylene and polyalkylene glycols, that act so as to provide acceptable viscosity, tactile qualities and retard drying. Formulations so composed have acceptable long-term interaction in vivo that render the acoustic media biocompatible with human tissue, organs and body fluids.

Gels of polyvinylpyrrolidone (PVP) can be prepared as thickened solutions in water, or alternatively in solution with plasticizers such as at least one of alkylene glycols and polyalkylene glycols, and/or fats and esters thereof, preferably containing two or greater carbon atoms and more preferably 2 to 6 carbon atoms, in a weight percent range of about 1% to about 80%, preferably about 10% to about 70%, and most preferred 20 to 60%. The preferred alkylene glycol comprises propylene glycol and the preferred polyalkylene glycol comprises polyethylene glycol. The most preferred is propylene glycol. In the case when a polyhydric alcohol is used, sorbitol is preferred. Propylene glycol is most preferred since it is in vivo biocompatible and biodegradable, and in the preferred embodiment, functions as a humectant to increase drying time, an antimicrobial and freeze inhibitor.

It is well known in the art to crosslink PVP so as to increase viscosity and modify the physical and mechanical properties. Such cross-linking techniques include high-energy radiation such as from e-beam and gamma sources and by chemically cross-linking, for example, with an amine containing polymers such as polyethyleneimine and chitosan. U.S. Pat. Nos. 5,306,504; 5,420,197 and 5,645,855 to Lorenz teach methods of cross-linking PVP using poly-functional amines. U.S. Pat. No. 6,379,702 to Lorenz et al. teaches production of cross-linked PVP with aqueous solutions of chitosan derivatives. Such cross-linked gels of PVP tend to be adhesive in nature and are commonly used as absorbent wound dressings and sealants.

The adhesivity and rheology of the cross-linked gels of Lorenz restrict free motion of instruments such as an ultrasound probe when in contact with such materials thus limiting acceptability of these formulations for ultrasound couplants or instrument lubricants. By comparison, the inventive gels composed of PVP, water and humectants are capable of being spread into a thin lubricous film aiding the free motion of an ultrasound transducer over an examination site.

A series of grades of PVP are commercially available from BASF Corporation, Mt. Olive, N.J., which differ as to purity and molecular weight. The BASF designation for pharmaceutical PVP is Kollidone and further described by K-value, which is an indicator of molecular weight. The range of molecular weights begins at 2,000-3,000 daltons for Kollidone K15; 40,000-54,000 daltons for Kollidone K30 and 360,000-1,500,000 daltons for Kollidone K90. The K-value is also indicative of the viscosity of a solution of a given percentage and increases as the K-value increases. The most preferred polymer grade for the device of this invention is Kollidone K90, due to its viscosity building capacity and rheology preferred for medical ultrasound imaging.

In the following examples, formulations of PVP in water and propylene glycol were prepared using Kollidone K30 and Kollidone K90 to access the viscosity values. Twenty weight percent of propylene glycol was added both as a humectant, to extend drying time and increase lubricity, and as an antimicrobial. A 10% formulation of Kollidone K90 in 70% de-ionized (DI) water and 20% propylene glycol yielded a viscosity of 480 centipoise (cps) whereas a 10% solution of Kollidone K30 was less than 100 centipoise (cps). Viscosities of this order are lower than required for efficient use as an ultrasound scanning gel. To increase the viscosity of the formulation, polymer concentrations were increased from 10 to 16% in a solution of 20% propylene glycol with the remainder being water.

### EXAMPLE 1

| | |
|---|---|
| Kollidone K90 | 10% |
| Propylene Glycol | 20% |
| De-ionized Water | 70% |
| Viscosity-Brookfield LVT Viscometer-#2 Spindle @ 1.5 rpm - | 440 cps |

### EXAMPLE 2

| | |
|---|---|
| Kollidone K90 | 16% |
| Propylene Glycol | 20% |
| De-ionized Water | 64% |
| Viscosity-Brookfield LVT Viscometer-#2 | 4000 cps |
| Spindle @ 1.5 rpm - | |

The increase of Kollidone K90 to 16% increased the viscosity to a more useful value, however, the gel was adhesive and stringy yielding unacceptable tactile properties.

### EXAMPLE 3

| | |
|---|---|
| Kollidone K90 | 16% |
| Propylene Glycol | 30% |
| De-ionized Water | 54% |
| Viscosity-Brookfield LVT Viscometer-#2 | 12,000 cps |
| Spindle @ 1.5 rpm - | |

It is known that as the weight percentage of plasticizer, such as propylene glycol is increased and the water content is decreased while maintaining polymer content at a constant, viscosity tends to increase and tactile qualities improve. To test viscosity and tactile quality effects, a third formulation, Example 3 above, was prepared based on 16% PVP Kollidone K90, propylene glycol 30% and the remainder de-ionized water. When compared to Example 2, the additional 10% of propylene glycol in Example 3 produced a viscosity of 12,000 cps, representing an increase of 8,000 cps from the 4,000 cps viscosity of Example 2. However, the product was paste-like and stringy. The viscosity increase with an additional 10% propylene glycol in the formulation indicated that, if the polymer concentration was lowered and the propylene glycol increased, the resultant product should have an acceptable viscosity and improved tactile qualities.

Additionally, Example 4 and Example 5 were prepared yielding viscosities of 3,200 and 7,700 cps respectively, while little improvement in tactile characteristics was noted.

### EXAMPLE 4

| | |
|---|---|
| Kollidone K90 | 12% |
| Propylene Glycol | 45% |
| De-ionized Water | 43% |
| Viscosity-Brookfield LVT Viscometer-#2 | 3,200 cps |
| Spindle @ 1.5 rpm - | |

### EXAMPLE 5

| | |
|---|---|
| Kollidone K90 | 12% |
| Propylene Glycol | 60% |
| De-ionized Water | 28% |
| Viscosity-Brookfield LVT Viscometer-#2 | 7,700 cps |
| Spindle @ 1.5 rpm - | |

To further evaluate the potential of polyethylene glycol to reduce the stickiness and minimize the formation of strings, the 12% PVP formula was modified to include polyethylene glycol (PEG). Samples at 12% PVP, that include the addition of PEG 300 and 8000 (molecular weights), respectively, are shown below.

### EXAMPLE 6

| | |
|---|---|
| Kollidone K90 | 12% |
| Propylene Glycol | 55% |
| PEG 300 | 5% |
| De-ionized Water | 28% |
| Viscosity-Brookfield LVT Viscometer-#2 | 6,900 cps |
| Spindle @ 1.5 rpm - | |

### EXAMPLE 7

| | |
|---|---|
| Kollidone K90 | 12% |
| Propylene Glycol | 55% |
| PEG 8000 | 5% |
| De-ionized Water | 28% |
| Viscosity-Brookfield LVT Viscometer-#2 | 7,760 cps |
| Spindle @ 1.5 rpm - | |

The above samples were heated to 100 degrees centigrade and held at temperature for 15 minutes, then cooled to room temperature prior to measurement of viscosity. Upon cooling, Example 7 containing PEG 8,000 precipitated and formed a cloudy solution whereas Example 6 containing PEG 300 remained stable at room temperature. Improved lubricity, drying time lack of stringiness was noted in both formulations.

To further evaluate the potential of producing useful gels at lower polymer concentrations, formulations containing 10% PVP Kollidone K90 were prepared as follows.

### EXAMPLE 8

| | |
|---|---|
| Kollidone K90 | 10% |
| Propylene Glycol | 55% |
| PEG 300 | 5% |
| De-ionized Water | 30% |
| Viscosity-Brookfield LVT Viscometer-#2 | 4,620 cps |
| Spindle @ 1.5 rpm - | |

### EXAMPLE 9

| | |
|---|---|
| Kollidone K90 | 10% |
| Propylene Glycol | 55% |
| PEG 8000 | 5% |
| De-ionized Water | 30% |
| Viscosity-Brookfield LVT Viscometer-#2 | 4,720 cps |
| Spindle @ 1.5 rpm - | |

Examples 10-12 below were prepared with PVP Kollidone K30 and K15. As can be seen, with 30% PVP K15 in Example 12 and the available water at 5%, the resultant viscosity is approximately six times less than in Example 8. Example 11 at 20% PVP K30 is about eight times less than Example 8 while Example 10 at 10% PVP K30 is nearly fifty times less than Example 8.

### EXAMPLE 10

| | |
|---|---|
| Kollidone K30 | 10% |
| PEG 300 | 5% |
| Propylene Glycol | 60% |
| De-ionized Water | 25% |
| Viscosity-Brookfield LVT Viscometer-#1 Spindle @ 30 rpm - | 95 cps |

### EXAMPLE 11

| | |
|---|---|
| Kollidone K30 | 20% |
| PEG 300 | 5% |
| Propylene Glycol | 60% |
| De-ionized Water | 15% |
| Viscosity-Brookfield LVT Viscometer-#1 Spindle @ 30 rpm - | 564 cps |

### EXAMPLE 12

| | |
|---|---|
| Kollidone K15 | 30% |
| PEG 300 | 5% |
| Propylene Glycol | 60% |
| De-ionized Water | 5% |
| Viscosity-Brookfield LVT Viscometer-#2 Spindle @ 12 rpm - | 720 cps |

Example 9 containing 5% PEG 8000 was unstable in solution as evidenced by cloudiness and precipitation. However, Example 8 remained stable after heating and cooling. All formulations were evaluated regarding, viscosity, lubricity, tack, string formation, adherence to ultrasound probe surfaces, and ease of removal from skin and instruments. Such evaluation indicated that Example 6 is preferred and Example 8 is the most preferred.

Polyvinyl alcohol also has potential use as in vivo biocompatible and bio-excretable ultrasound couplants. Such couplant gels that can be prepared by several methods including, PVA in mixtures of water composed of glycerol, ethyl alcohol, ethylene and propylene glycol, polyglycols, polyhydric alcohols, dimethyl formamide and acetamine. The gels which form are thought to be the result of hydrogen bonding. A second method involves cross-linking by reaction with organic and inorganic compounds. PVA can be cross-linked by di-functional compounds that condense with organic hydroxyl groups such as gluteraldehyde, acetaldehyde, formaldehyde and monoaldehydes, maleic and oxalic acid, dimethyl urea, glyoxal, triethylomelamine, hydrochloric acid, polyacrolein, diisocyanates, divinyl sulfate, and ceric redox systems.

PVA cross-linked gels can also be formed by exposure to ultraviolet energy in the presence of photo-initiators such as chromium compounds and by exposure to ionizing radiation from e-beam and gamma ray sources. However, gels formed by these methods tend to be cohesive not easily spread into thin films as generally required for medical ultrasound procedures.

The production method preferred for preparation of PVA gels of the inventive device are formulations of PVA in alkylene and/or polyalkylene glycols and water solutions.

The following examples illustrate compositions and formulations that can be used to prepare PVA gels suitable for use in medical ultrasound procedures. Polyvinyl alcohol (PVA) used in these formulations is commercially available from suppliers such as Spectrum, Auburn, Wash., (sold under the name Povidone) as the ethenol homopolymer: (CH₂CHOH)ₙ, having a degree of hydrolysis between 85-99%.

One method of gel formation utilizes inorganic and organic compounds for cross-linking to effect viscosity increase of the base PVA solution is demonstrated.

### EXAMPLE 13

A 10% solution of PVA (Spectrum 85-89% hydrolysis) and a 1% solution of sodium tetraborate were prepared for gel production by cross-linking. To 100 grams of 10% PVA, 7 grams of 1% sodium borate was added while stirring. Cross-linking occurred immediately forming a viscous, cohesive mass that due to its rheology was unsuitable for use as an ultrasound gel.

### EXAMPLE 14

To 100 grams of 10% PVA, 10 grams of propylene glycol was first added, followed by drop-wise addition of 10 grams of 1% sodium borate while stirring. The solution thickened without clumping. The initial viscosity of the 10% PVA solution was approximately 2,800 cps as measured on a Brookfield LVT Viscometer using a #2 Spindle at 1.5 RPM. After a period of 48 hours, cross-linking had occurred as evidenced by an increase in viscosity to 8,840 cps and formation of a cohesive, elastic gel that was unsuitable for general ultrasound imaging purposes.

### EXAMPLE 15

To 100 grams of 10% PVA, 15 grams of 4% gluteraldehyde was added while stirring. Immediate thickening or evidence of cross-linking was not observed. The viscosity of the 10% PVA solution prior to addition of gluteraldehyde was approximately 2,800 cps. After a period of 48 hours, the viscosity increased to 9,320 cps as measured above in Example # 2. The gel which formed was cohesive and elastic, and generally unsuitable for general ultrasound imaging.

Data from earlier observations indicated that PVA solutions of 45% in water produced thick flowable gels. However, such water based gels dried and quickly became tacky. To improve drying characteristics, propylene glycol, polyethylene glycol, PEG and glycerin were added to separate formulations to perform as humectants. Conclusions drawn from the experimental data indicate that the most preferred humectant is propylene glycol, followed by PEG 300 glycerol and sorbitol. The concentration of the preferred humectant, propylene glycol, was determined to be 20% of the formulation since 20% and greater weight percentages of propylene glycol slows the drying time, reduces tack, and acts as an anti-microbial.

The concentration of polymer required to achieve a target viscosity of 15,000 cps was determined to be in a range of 6 to 25%, 10% PVA being most suitable. For example, a formulation that contains 10% PVA, 20% propylene glycol, the remainder being water, produces a solution viscosity of 5,000 cps whereas, a formulation consisting of 15% PVA, 20% propylene glycol, the remainder water, yielded a viscosity of approximately 52,600 cps, when measured as in Example # 2.

### EXAMPLE 16

| | |
|---|---|
| Polyvinyl Alcohol | 15% |
| Propylene Glycol | 20% |
| Water (WFI) | 65% |

(WFI = Water For Injection)

### EXAMPLE 17

| | |
|---|---|
| Polyvinyl Alcohol | 10% |
| Propylene Glycol | 20% |
| Water (WFI) | 70% |

(WFI = Water For Injection)

The present invention also contemplates mixtures of PVP and PVA as shown in the following Examples 18 and 19 which illustrate viscosity and tactile properties. Example 19 shows acceptable properties while Example 18, which contains the lower molecular weight Kollidone 30, exhibits a lower viscosity and is less acceptable for use.

### EXAMPLE 18

| | |
|---|---|
| Polyvinyl Alcohol | 9% |
| Kollidone 30 | 3% |
| Propylene Glycol | 20% |
| De-ionized Water | 68% |
| Viscosity-Brookfield LVT Viscometer-#2 Spindle | 3,700 cps |
| @ 1.5 rpm - | |

### EXAMPLE 19

| | |
|---|---|
| Polyvinyl Alcohol | 9% |
| Kollidone 90 | 3% |
| Propylene Glycol | 20% |
| De-ionized Water | 68% |
| Viscosity-Brookfield LVT Viscometer-#2 | 6,420 cps |
| Spindle @ 1.5 rpm - | |

The formulations of PVP and PVA were compared with regard to viscosity, lubricity, tack, string formation, adherence to ultrasound probe surfaces and ease of removal from the skin and instruments. Example 16 containing 15% PVA was preferred whereas Example 6 which contains 12% PVP Kollidone K90, 55% propylene glycol, 5% PEG 300 and the balance water was more preferred. The most preferred formulation is Example 8 which contains 10% PVP, 5% PEG 300, 55% propylene glycol and the remainder water.

For use as in vivo biocompatible ultrasound couplants, the gels of PVA and PVP must be sterilized. The common and acceptable sterilization methods of e-beam and gamma irradiation are unsuitable for polyvinyl alcohol formulations. Radiation dosages prescribed for terminal sterilization protocols, generally 25 Kilograys (KGY) and above, are sufficient to cross-link or cause chain sission leading to changes in rheology and viscosity of the solutions. Such response to high energy exposure decreases lubricity and changes flow behavior by creating insoluble solids and cohesive masses that are not easily spread into a thin film or layer between the active face of an ultrasound probe and skin, or the ionizing energy can break the polymer bonds, thus reducing the viscosity. In either case, the products of high energy radiation are unsuitable for ultrasound imaging procedures when thin, flowable films are desired. As an example, U.S. Pat. No. 5,405,366 to Fox et al. teaches methods to produce cross-linked polyethylene in combination with other compounds such as PVA and gylcols, by subjecting formulations of these compounds to high-energy radiation sufficient to form cross-linked compounds that are non-stringy and cohesive. Such cross-linked compounds could be used for ultrasound standoffs or as attachments; however lack the physical properties preferred for use as ultrasound couplants and lubricants.

The present invention describes, non cross-linked solutions of PVP and/or PVA, water, alkylene and/or polyalkylene glycols which are sterilized by heat to avoid cross-linking. Given the constraints related to the cross-linking characteristics of PVA and PVP, post-production sterilization of the final package by high-energy sources is not practical. Viable alternatives to conventional post production high energy sterilization methods include sterilization of the finished formulation in bulk form using autoclave protocols, followed by aseptic filling and packaging, or heat sterilization of the entire package in its final form.

In one example of manufacture, the base polymer solution is compounded in a reactor vessel suitable for vacuum degassing and heating the solution. PVA, PVP, or blends thereof, are dissolved in pyrogen free water and polyalkylene glycols with by heating and stirring, then vacuum degassed, nitrogen backfilled and heated to 80° C. Once the polymers are completely in solution, the gel is cooled for packaging into suitable containers for sterilization in final form, according to conventional steam sterilization protocols.

An alternative to post packaging sterilization required by the method of the previous example, integrates production and sterilization of the polymer solution by use of a reactor vessel suitable for compounding, vacuum degassing and heating the solution under pressure, to a core temperature of 121° C. for sterilization. In practice, the polymer is compounded in pyrogen free water alone or alternatively with alkylene and/or polyalkylene glycols, and blends thereof, and degassed under vacuum at 60° C. While under seal, the reactor vessel is back-filled with nitrogen gas to 1 atmosphere. The formulation is heated to a core temperature of 121° C., held for 15 to 30 minutes at temperature and while stirring allowed to cool below 100° C. to a temperature suitable to aseptic packaging.

The polyvinylpyrrolidone and polyvinyl alcohol gels of the present invention, are not intended for, nor can perform as stand-alone attachment to an ultrasound probe or acoustic standoff. The inventive gels are flowable, lubricous, capable of forming thin films between the transducer face and examination site, and lack the structural rigidity of the device of Hayakawa.

The inventive couplant fluids or gels, being in vivo biocompatible and bio-eliminated, can remain in the body without harming such since they are subsequently excreted from the body after being eroded, metabolized or absorbed via natural pathways and processes. In sterile form, the inventive in vivo biocompatible ultrasound couplants provide utility and safety for use when ultrasound examinations are performed in contact with organs, tissue and body fluids.

For use in intraoperative and intracavity procedures, the inventive couplant is placed inside a protective cover and in contact with the probe face to couple the acoustic energy between the active area of the probe, the ultrasound transducer, and the cover or sleeve. Since during a surgical or intracavity ultrasound examination or therapeutic procedure, the external surface of the probe cover is in contact with body fluids that naturally conduct acoustic energy, additional couplant on the external surface of the probe cover is seldom required. In the event of accidental rupture of the protective cover, introduction of the inventive ultrasound couplant into the body cavity can result in its contact with tissue, organs and fluids. Should such an event occur, the couplants of this inventive device will not adversely affect the patient due to its biocompatibility and bio-elimination in vivo.

For patient comfort during intracavity, i.e. vaginal, rectal and transesophageal ultrasound examinations or therapeutic procedure, a lubricant such as the inventive device is often required on the exterior of the transducer protective probe cover or the endoscope shaft prior to introduction into a body cavity. In instances when such in vivo biocompatible couplants are used for transcutaneous scanning or therapy, ophthalmic imaging or ultrasound guided needle punctures, such as amniocentesis and transcutaneous biopsy procedures, additional couplant is generally required to couple sound between the external surface of the protective cover or sleeve and the patient. Such couplant is usually placed on the skin of the patient in the examination area.

In instances where an ultrasound probe is covered by a protective sheath, as previously mentioned, the ultrasound couplants of the inventive device provide not only acceptable acoustic coupling properties when such couplant is placed on the outside of the protective sheath, but also when placed within the sheath (i.e. between the active face of the ultrasound probe and the sheath).

The hydrophilic polymeric compounds, PVP and PVA, meet the objectives of in vivo biocompatibility and elimination from the body by natural pathways and processes. These polymers are formulated with water, preferably pyrogen free water, and optionally further including at least one of alkylene glycol and polyalkylene glycol in concentrations by weight between 1.0 and 80% by weight. When prepared in final form, such mixtures exhibit acoustic properties similar to that of human tissue, render acceptable low levels of artifact, distortion and attenuation of the ultrasound energy, and acceptable viscosity, film forming and adherence characteristics.

While the invention has been described with reference to preferred embodiments it is to be understood that the invention is not limited to the particulars thereof. The present invention is intended to include process, formulation and modifications which would be apparent to those skilled in the art to which the subject matter pertains without deviating from the spirit and scope of the appended claims.

1. An in vivo biocompatible ultrasound acoustic couplant or lubricant comprising:
at least one of 3-30 wt. % polyvinylpyrrolidone and 6-25 wt. % polyvinyl alcohol;
at least one of (alkylene glycol, polyalkylene glycol, and fats and esters thereof in an amount of 1-80 wt. %; and,
the balance water.

2. The couplant or lubricant of claim 1 wherein said at least one of (alkylene glycol, polyalkylene glycol, and fats and esters thereof is included in an amount of 10-70 wt. %.

3. The couplant or lubricant of claim 1 wherein said at least one of (alkylene glycol, polyalkylene glycol, and fats and esters thereof is included in an amount of 20-60 wt. %.

4. The couplant or lubricant of claim 1 wherein the molecular weight of polyvinylpyrrolidone is in the range of 360,000-1,500,000 daltons.

5. The couplant or lubricant of claim 1 wherein polyvinylpyrrolidone is included in an amount of 10-20 wt. %.

6. The couplant or lubricant of claim 1 wherein polyvinylpyrrolidone is included in an amount of 10-12 wt. %.

7. The couplant or lubricant of claim 1 wherein polyvinyl alcohol is included in an amount of 9-15 wt. %.

8. The couplant or lubricant of claim 1 wherein said alkylene glycol comprises propylene glycol.

9. The couplant or lubricant of claim 1 wherein said polyalkylene glycol comprises polyethylene glycol.

10. The couplant or lubricant of claim 1 wherein the polyethylene glycol has a molecular weight of 300.

11. The couplant or lubricant of claim 1 wherein said at least one of (alkylene glycol, polyalkylene glycol, and fats and esters thereof comprises two or more carbon atoms.

12. The couplant or lubricant of claim 11 wherein said at least one of (alkylene glycol, polyalkylene glycol, and fats and esters thereof comprises 2-6 carbon atoms.

13. The couplant or lubricant of claim 1 comprising:
15 wt. % polyvinyl alcohol;
20 wt. % propylene glycol; and,
the balance water.

14. The couplant or lubricant of claim 1 comprising:
12 wt. % polyvinylpyrrolidone;
55 wt. % propylene glycol;
5 wt. % polyethylene glycol;
the balance water.

15. The couplant or lubricant of claim 1 comprising:
10 wt. % polyvinylpyrrolidone;
55 wt. % propylene glycol;
5 wt. % polyethylene glycol;
the balance water.

16. The couplant or lubricant of claim 1 comprising:
9 wt. % polyvinyl alcohol
3 wt. % polyvinylpyrrolidone;
20 wt. % propylene glycol;
the balance water.

17. The couplant or lubricant of claim 1 being non cross-linked.

18. The couplant or lubricant of claim 1 being sterilized.

19. The couplant or lubricant of claim 1 being in the form of a liquid or gel.

20. The couplant or lubricant of claim 1 wherein said water is pyrogen free water.

21. The couplant or lubricant of claim 1 wherein the molecular weight of polyvinylpyrrolidone is in the range of 40,000-54,000 daltons.
